(19) United States
(12) Patent Application Publication
(10) Pub. No.: US 2020/0294653 A1
(43) Pub. Date: Sep. 17, 2020

(54) SPECTRAL (MULIT-ENERGY) IMAGING VISUALIZATION

(71) Applicant: KONINKLIJKE PHILIPS N.V., EINDHOVEN (NL)

(72) Inventor: SHLOMO GOTMAN, HAIFA (IL)

(21) Appl. No.: 16/644,521

(22) PCT Filed: Aug. 31, 2018
(86) PCT No.: PCT/EP2018/073491

§ 371 Date: Mar. 5, 2020

Related U.S. Application Data
(60) Provisional application No. 62/555,786, filed on Sep. 8, 2017.

(51) Int. Cl. G16H 30/20; G06T 11/00; G16H 30/40; G01J 3/28

(52) U.S. Cl. CPC G16H 30/20; G06T 11/003; G16H 30/40; G01J 2003/2826; G06T 2207/10081; G06T 2207/10036; G01J 3/2823

(57) ABSTRACT

A computing system (126) includes a memory device (130) configured to store image visualization application software, spectral imaging data, and spectral image reconstruction algorithms. The application software is configured to read electronic files containing images and formatted in a first format, the imaging data is formatted in a second different format, which the application software cannot read and/or interpret, and the algorithms are configured to read electronic files formatted in the second different format. A processor (128) is configured to access at least one of the algorithms through a proprietary software interface and process the imaging data with the at least one of the algorithms to produce a spectral image, and execute the application software to construct a graphical user interface with an image viewport displaying the spectral image. A display (132) is configured to display the graphical user interface with the spectral image displayed in the viewport.

### FIELD OF THE INVENTION

The following generally relates to visualizing spectral (multi-energy) imaging data from one vendor with a visualization device of a different vendor, and is described with particular application to visualizing the spectral imaging data with a picture archive and communication system (PACS) of the different vendor, where application software of the PACS cannot read and/or interpret the format of the spectral imaging data.

### BACKGROUND OF THE INVENTION

Spectral (or multi-energy) computed tomography (CT) imaging can be implemented in a few different ways. For example, one implementation includes a CT scanner with a broadband x-ray tube and an energy-resolving, multi-layer detector such as a dual-layer detector where lower energy photons are absorbed and detected in a top scintillator layer, and higher energy photons traversing the top scintillator layer are absorbed and detected in a bottom scintillator layer, which is under the top layer relative to a direction of impinging x-ray radiation. Another implementation includes a CT scanner configured with kVp switching (e.g., 80 kVp, 140 kVp, etc.). Another implementation includes a CT scanner configured with multiple x-ray tubes. Another implementation includes a CT scanner with direct conversion photon counting detectors (e.g., CZT, etc.). Another implementation includes a filter(s) made of different materials inserted into the X-Ray beam. Another implementation includes two or more consecutive scans at the same location with different kVp values

Such implementations can provide several different types of spectral imaging data. For instance, the imaging data can include wide spectrum attenuation values (i.e. “conventional” or non-spectra. CT), attenuation values in different x-ray energy bands, virtual monochromatic images, etc. Where a contrast agent is used (e.g. iodine, gadolinium, etc.), a contrast agent quantitative map and/or virtual non-contrast (VNC) images can be derived. The contrast agent quantitative map can show, e.g., the local concentration of iodine (e.g., in mg/ml). The VNC image shows a virtual (calculated) image which resembles a conventional CT image without administration of a contrast agent. Another approach calculates a volumetric Z-effective map, which estimates a mean atomic number of the material compound in each image voxel. Other approaches generate volumetric maps for materials such as calcium, uric-acid, etc.

The reconstruction algorithms employed to generate spectral images generally include vendor proprietary algorithms based on the vendor-specific imaging data generated by the vendor's CT imaging system. As a consequence, the spectral images generally can only be generated by proprietary hardware and/or software of the vendor, such as the CT imaging system itself or by a processing workstation of the vendor. However, diagnostic reading is routinely performed on a product of a different vendor such as a picture archive and communication system (PACS), which is unable to read the spectral imaging data from the imaging system. A solution has included integrating a special spectral on the PACS, and running the special spectral application when spectral functionality is desired to generate spectral images. This special spectral application is not part of the PACS application software, but can be executed within the PACS environment as a plug-in or the like.

Unfortunately, this approach has several limitations. For example, the user interface (UI) of the spectral application is different from the UI of the PACS. Thus, the user has to become familiar with different interfaces and operation. Furthermore, leaving the PACS application software and running the special spectral application disrupts the normal workflow of reading images. In addition, the imaging data used to generate results is usually stored on the PACS itself. Thus, the special spectral application has to either access the PACS database using a relatively slow digital imaging and communications in medicine (DICOM) interface or also keep a copy of all the spectral imaging data on its own storage. Furthermore, a PACS system, unlike the special spectral application, usually has direct and optimized access to its own database for faster performance.

### SUMMARY OF THE INVENTION

Aspects described herein address the above-referenced problems and others.

In one aspect, a computing system includes a memory device configured to store image visualization application software, spectral imaging data, and spectral image reconstruction algorithms. The image visualization application software is configured to read electronic files containing images and formatted in a first format. The spectral imaging data is formatted in a second different format, which the image visualization application software cannot read and/or interpret. The spectral image reconstruction algorithms are configured to read electronic files formatted in the second different format. The computing system further includes a processor configured to access at least one of the spectral image reconstruction algorithms through a proprietary software interface and process the spectral imaging data with the at least one of the spectral image reconstruction algorithms to produce a spectral image. The processor is further configured to execute the image visualization application software to construct a graphical user interface with an image viewport displaying the spectral image. The computing system further includes a display configured to display the graphical user interface with the spectral image displayed in the viewport.

In another aspect, a computer readable medium is encoded with computer executable instructions, which, when executed by a processor of a computer, cause the processor to: receive spectral image reconstruction algorithms and spectral imaging data, access at least one of the spectral image reconstruction algorithms through a proprietary software interface, process the spectral imaging data with the at least one of the spectral image reconstruction algorithms to produce a spectral image, execute image visualization application software to construct a graphical user interface with an image viewport displaying the spectral image, where the image visualization application software cannot read and/or interpret the spectral imaging data, and display the graphical user interface with the spectral image displayed in the viewport.

In another aspect, a method includes receiving spectral image reconstruction algorithms and spectral imaging data. The method further includes receiving proprietary spectral image reconstruction algorithms and proprietary spectral imaging data at a picture archive and communication system. The method further includes accessing, with the picture archive and communication system, at least one of the proprietary spectral image reconstruction algorithms only through a proprietary software interface. The method further includes processing, with the picture archive and communication system, the proprietary spectral imaging data with the at least one of the proprietary spectral image reconstruction algorithms to produce a spectral image. The method further includes executing, with the picture archive and communication system, image visualization application software of the picture archive and communication system to construct a graphical user interface with an image viewport displaying the spectral image. The image visualization application software cannot read and/or interpret a format of the proprietary spectral imaging data. The method further includes displaying the graphical user interface with the spectral image displayed in the viewport.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 schematically illustrates an example system including an imaging system configured for spectral imaging and a PACS configured to visualize spectral images generated at the PACS with spectral imaging data from the imaging system.

FIG. 2 schematically illustrates an example spectral image type menu presented by the PACS in a GUI.

FIG. 3 schematically illustrates an example energy level dialog box, presented by the PACS in the GUI, with a list of user selectable image reconstruction energy levels for a mono-energetic spectral image type.

FIG. 4 schematically illustrates an example energy level dialog box, presented by the PACS in the GUI, with a character input box for user entry of an image reconstruction energy level for a mono-energetic spectral image type.

FIG. 5 schematically illustrates an example energy level dialog box, presented by the PACS in the GUI, with a character input box for user entry of an image reconstruction energy level and a character input box for user entry of an energy level increment for a mono-energetic spectral image type.

FIG. 6 schematically illustrates a slider widget, presented by the PACS in the GUI, for selecting an energy level for image reconstruction for a mono-energetic spectral image type.

FIG. 7 schematically illustrates a knob widget, presented by the PACS in the GUI, for selecting an energy level for image reconstruction for a mono-energetic spectral image type.

FIG. 8 schematically illustrates the GUI with a single viewport displaying a single spectral image.

FIG. 9 schematically illustrates the GUI with the single viewport displaying multiple spectral images in a split screen format.

FIG. 10 schematically illustrates the GUI with the single viewport displaying multiple overlapping spectral images.

FIG. 11 schematically illustrates the GUI with multiple viewports, each displaying a single spectral image.

FIG. 12 schematically illustrates the GUI with multiple viewports where at least one viewport displays multiple overlapping spectral images.

FIG. 13 illustrates an example method in accordance with an embodiment herein.

### DETAILED DESCRIPTION OF EMBODIMENTS

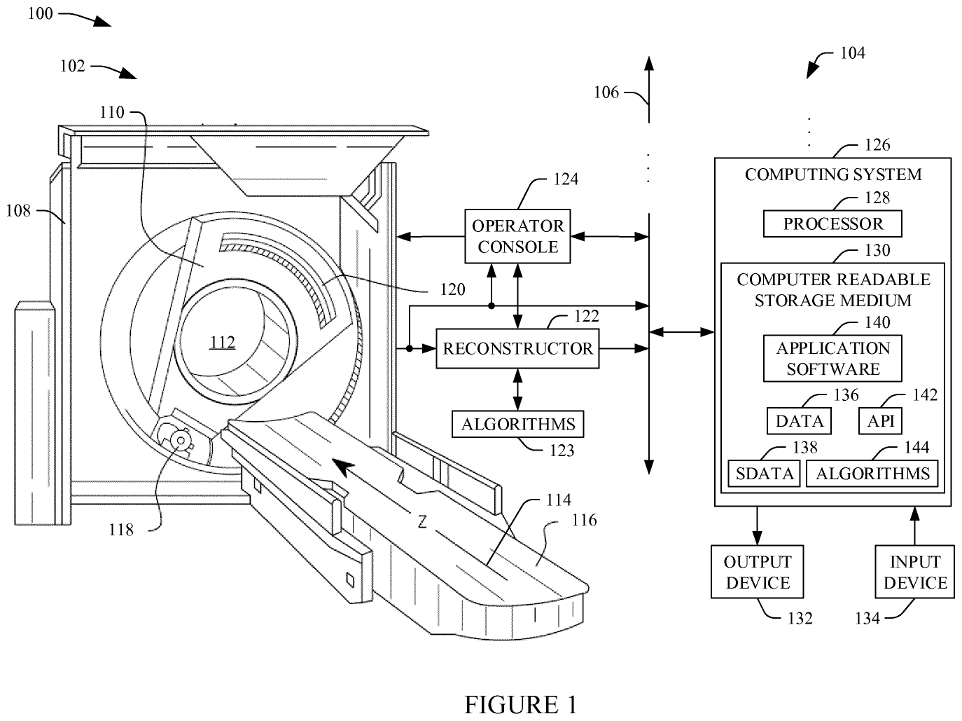

FIG. 1 schematically illustrates a system 100 comprising an imaging system 102, one or more other devices 104, and a communication path 106 (e.g., a network, a bus, a direct connection, etc.).

The imaging system 102 and the one or more other devices 104 communicate with each other over the communication path 106. Patient data such as imaging and/or non-imaging data are conveyed over the communication path 106 via Digital Imaging and Communications in Medicine (DICOM), Health Level 7 (HL7), and/or other communication protocol. In this example, a vendor of the imaging system 102 is different from a vendor(s) of the one or more other devices 104, and application software of the vendor(s) of the one or more other devices 104 loaded on and executed by the one or more other devices 104 cannot read and/or interpret a format of or process the spectral imaging data from the imaging system 102.

The illustrated imaging system 102 includes a computed tomography (CT) scanner configured for non-spectral and spectral (multi-energy) imaging. The imaging system 102 includes a stationary (i.e., non-rotating) gantry 108 and a rotating gantry 110. The rotating gantry 110 is rotatably supported by the stationary gantry 108 and rotates around an examination region 112 about a longitudinal or z-axis 114. A subject support 116, such as a couch, supports an object or subject in the examination region. The subject support 116 is movable in coordination with performing an imaging procedure so as to guide the subject or object with respect to the examination region 112 for loading, scanning, and/or unloading the subject or object.

A radiation source 118, such as an x-ray tube, is rotatably supported by the rotating gantry 110. The radiation source 118 rotates with the rotating gantry 110 and emits X-ray radiation that traverses the examination region 112. In one embodiment, the radiation source 118 is a single x-ray tube configured to emit broadband (polychromatic) radiation for a single selected peak emission voltage (kVp) of interest (i.e. the energy spectrum at that kVp). In another embodiment, the radiation source 118 is configured to switch between at least two different emission voltages (e.g., 70 keV, 100 keV, etc.) during scanning. In yet another embodiment, the radiation source 118 includes two or more x-ray tubes angular offset on the rotating gantry 110 with each configured to emit radiation with a different mean energy spectrum. In still another embodiment, the radiation source 118 includes a combination thereof and/or other spectral imaging approach. U.S. Pat. No. 8,442,184 B2 describes a system with kVp switching and multiple x-ray tubes, and is incorporated herein by reference in its entirety.

A radiation spectrum sensitive detector array 120 subtends an angular arc opposite the radiation source 118 across the examination region 112. The detector array 120 includes one or more rows of detectors that arranged with respect to each other along the z-axis 114 direction and detects radiation traversing the examination region 112. In one embodiment, the detector array 120 includes an energy-resolving detector such as a multi-layer scintillator/photo-sensor detector (e.g., U.S. Pat. No. 7,968,853 B2, which is incorporated herein by reference in its entirety) and/or a photon counting (direct conversion) detector (e.g., WO2009072056A2, which is incorporated herein by reference in its entirety). With an energy-resolving detector, the radiation source 118 includes the broadband, kVp switching and/or multiple X-ray tube radiation source. In another embodiment, the detector array 120 includes a non-energy-resolving detector, and the radiation source 118 includes the kVp switching and/or the multiple X-ray tube radiation source. The detector array 120 generates spectral projection data (line integrals) indicative of the different energies. A reconstructor 122 reconstructs the spectral projection data with one or more different reconstruction algorithms 123, including a spectral reconstruction algorithm(s) and a non-spectral reconstruction algorithm(s). The non-spectral reconstruction algorithm(s) produces conventional broadband (non-spectral) volumetric image data, e.g., by combing the spectral projection data and reconstructing the combined volumetric image data and/or combining spectral images. The spectral reconstruction algorithm(s) produces basis volumetric image data, e.g., first basis volumetric image data, second basis volumetric image data, . . . , Nth basis volumetric image data. For example, for dual energy, the reconstructor 122 can generate high/low energy data sets, photo-electric effect/Compton scatter volumetric image data sets, calcium/iodine volumetric image data sets, bone/soft tissue volumetric image data sets, etc. Other data sets include mono-energetic/monochromatic volumetric, calcium suppression, effective Z (atomic number), electron density, iodine density, contrast-enhanced structures, iodine suppression, uric acid, uric acid suppression, virtual non-contrast, K-edge, etc. data sets.

An operator console 124 allows an operator to control an operation of the system 102. This includes selecting an imaging acquisition protocol (e.g., multi-energy), selecting a reconstruction algorithm (e.g., multi-energy), invoking scanning, etc. The operator console 124 includes an output device(s) such as a display monitor, a filmer, etc., and an input device(s) such as a mouse, keyboard, etc.

The spectral projection data and/or spectral volumetric image data (collectively referred to as spectral imaging data herein) can be stored in a hardware memory device of the imaging system 102, such as a hardware memory device of the console 124, a hardware memory device of the reconstructor 122, and/or other hardware memory. The spectral imaging data can additionally, or alternatively, be transferred via the communications path 106 to the one or more other devices 104, e.g., from the console 124 and/or reconstructor 122, or first formatted in a format of the vendor of the imaging system 102, such as a spectral base image (SBI) DICOM format, and then transferred via the communications path 106 to the one or more other devices 104.

The illustrated one or more other devices 104 includes at least a computing system 126. Other example devices include another imaging system (e.g., CT, PET, SPECT, MR, etc.), a remote display, a data repository (e.g., a radiology information system (RIS), a hospital information system (HIS), etc.), an electronic medical record (EMR), and/or other device. In this example, the computing system 126 includes a visualization system such as a picture and archiving system (PACS). In another embodiment, the computing system 126 includes another type of visualization system. In yet another embodiment, the computing system 126 includes an image analyzing/processing system.

The illustrated PACS 126 includes at least a processor 128 (e.g., a microprocessor, a central processing unit, etc.) and a computer readable storage medium 130, which excludes transitory medium and includes physical memory and/or other non-transitory medium. The PACS 126 further includes an output device(s) 132 such as a display monitor, a filmer, etc., and an input device(s) 134 such as a mouse, keyboard, touchscreen, etc. The processor 128 is configured to execute at least one computer readable instruction stored in the computer readable storage medium 130. The processor 128 may also execute one or more computer readable instructions carried by a carrier wave, a signal or other transitory medium. The computer readable storage medium 130 includes data memory (“data”) 136 for storing imaging data formatted in a predetermined (e.g., standard) DICOM format, and spectral data memory (“sdata”) 138 for storing spectral imaging data from the imaging system 102 and/or spectral imaging data formatted in the SBI DICOM format. The data 136 and sdata 138 can be part of a same or different memory region.

The at least one computer readable instruction includes application software 140. The application software 140 includes visualization software of the vendor of the PACS 126. This software includes a graphical user interface with one or more viewports for displaying images, a software menu for selecting and loading an image(s) into a viewport(s), software image manipulation tools such as zoom, pan, rotate, fuse, color, etc., software image measurement tools such as tools to measure a standard deviation of a region of interest, a distance between points, etc., and/or other visualization features. With these features, the PACS 126 can read and visualize images stored in the data 136 (i.e., images formatted in a standard DICOM format). The application software 140 uses an application programming interface (API) 142 or the like for executing calls to spectral reconstruction algorithms 144 to process the spectral imaging data stored in the sdata 138 and generate spectral images. Without the API 142 and the algorithms 144, the computing system 126 cannot read, interpret or process the spectral imaging data stored in the sdata 138.

The spectral reconstruction algorithms 144 include algorithms of, and provided by, the vendor of the imaging system 102, and not the vendor of the PACS 126. In this example, the spectral reconstruction algorithms 144 includes all of or a subset of the reconstruction algorithms 123 of the reconstructor 122. For example, in this example the spectral reconstruction algorithm(s) include the algorithm(s) that produces basis volumetric image data, e.g., first basis volumetric image data, second basis volumetric image data, . . . , Nth basis volumetric image data. For example, for dual energy, the reconstructor 122 can generate high/low energy data sets, photo-electric effect/Compton scatter volumetric image data sets, calcium/iodine volumetric image data sets, bone/soft tissue volumetric image data sets, etc. Other data sets include mono-energetic/monochromatic volumetric, calcium suppression, effective Z (atomic number), electron density, iodine density, contrast-enhanced structures, iodine suppression, uric acid, uric acid suppression, virtual non-contrast, K-edge, etc. data sets.

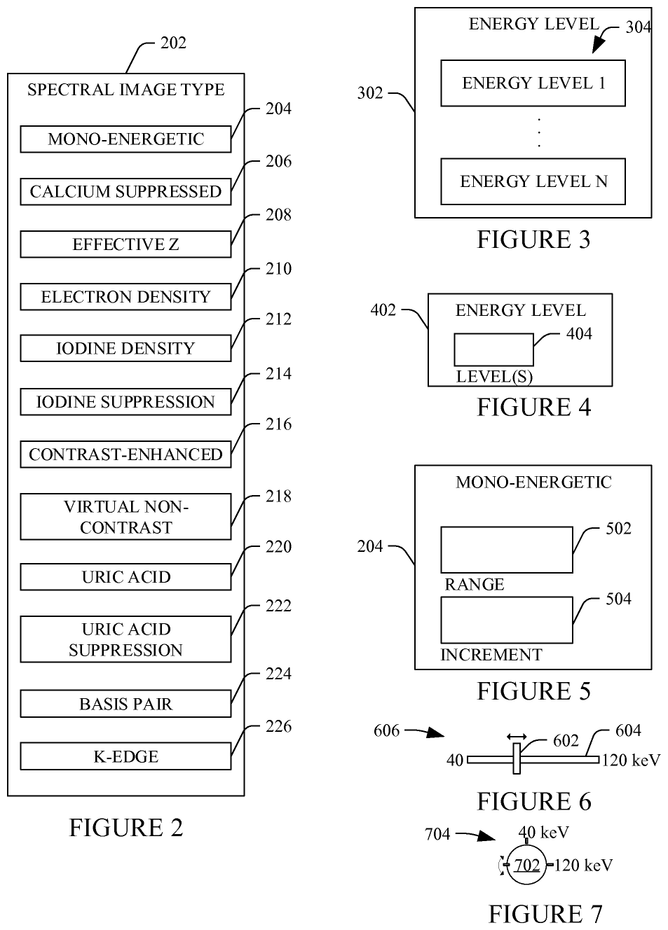

In one instance, the application software 140 of the vendor of the PACS 126 constructs and visually presents a GUI via a display of the output device 132. The GUI includes a list (e.g., a drop-down menu, a pop-up menu, and/or other type of menu) that visually presents graphical control elements/widgets (e.g., a list box) that allow a user to choose or select (e.g., via mouse click, touch, etc.) a type of spectral image to generate from a list of available type of spectral images to generate. FIG. 2 shows an example drop-down menu 202 including a list of widgets, each corresponding to a different type of spectral image to generate, including mono-energetic 204, calcium suppression 206, effective Z (atomic number) 208, electron density 210, iodine density 212, iodine suppression 214, contrast-enhanced 216, virtual non-contrast 218, uric acid 220, uric acid suppression 222, basis pair 224, and K-edge 226. In one instance, selecting (via mouse, touchscreen, etc.) one of the widgets in the menu 202 calls the corresponding spectral reconstruction algorithm from the algorithms 144 to read the spectral imaging data and generate a spectral image of that type.

In another instance, additional information may be required before a spectral image is generated. For example, for a mono-energetic image, at least one value (e.g., 100 keV) indicating an x-ray energy level of interest may have to be entered, e.g., where a default or predetermined value is not used. For this, selecting the mono-energetic widget 204 may invoke display of a dialog box with a list of energies to choose from and/or a character input box for entering one or more values of interest. FIG. 3 shows a dialog box 302 with a list 304 of energy level widgets (e.g., 40, 70, 100 and 120 keV) from which the user selects one. FIG. 4 shows a dialog box 402 with a character input box 404 for entry of an energy level(s). Alternatively, the mono-energetic widget 204 itself may include the character input box 404. In yet another example, the user can select and/or enter a range of energy levels, e.g., 20-200 keV, and an energy level increment, e.g., 1, 5, 10, 25, etc. keV, where an image is generated over the range at multiples of the increment. FIG. 5 shows the mono-energetic widget 204 with a character input box 502 for a range and an increment box 504 for an increment. In still another example, information in a header or elsewhere of the image electronic file is used to automatically identify an energy value(s).

The above provided examples for the Mono-energetic widget 204. It is to be appreciated that one or more of the other widgets 206-226 may also require or optionally receive input before generating a spectral image. Examples are provided next. For the Calcium Suppression widget 206, the additional information may include a suppression level such as 50-100. For the Effective Z widget 208, the additional information may include selecting a color map (user-selectable and/or adjustable) to apply. For the Electron Density widget 210, the additional information may likewise include selecting a color map (user-selectable and/or adjustable) to apply. For the Iodine Density widget 212, the additional information may indicate the image is to be fused with (or display as color overlay on top of) a non-spectral and/or mono-energetic image. For the Contrast-Enhanced widget 216, the additional information may indicate an enhancement level such as 1.1-1.5. For the Uric Acid widget 220, the additional information may indicate the image is to be fused with (or display as color overlay on top of) a non-spectral and/or mono-energetic image. It is to be understood that the above examples are non-limiting examples, and other types of spectral images and/or inputs are contemplated herein.

The GUI is native to the PACS 126 in that the application software 140 constructing and presenting the GUI is from the vendor of the PACS. The menu 202 is also native to the PACS 126 in that the application software 140 constructing and presenting the menu 202 is from the vendor of the PACS 126. The spectral reconstruction algorithms 144 are not native to the PACS 126 as they are algorithms of a vendor other than the vendor of the PACS 126 (e.g., the vendor of the imaging system 102). Without the API 142, the application software 140 cannot make calls to the spectral reconstruction algorithms 144, and without the spectral reconstruction algorithms 144, the PACS 126 cannot process the spectral imaging data. The spectral reconstruction algorithms 144 can read and process the spectral imaging data stored in the sdata 138 and/or the spectral imaging data formatted in the SBI DICOM format and stored in the sdata 138. The application software 140 may also present other visualization features such as presets for setting an attenuation or Hounsfield unit window and level for bone, lung, soft tissue, etc., and these presets can be used with the imaging data stored in the data 136 and the spectral imaging data stored in the sdata 138. The menus 202 are also native to the PACS 126 in that the application software 140 constructing and presenting the menus are from the vendor of the PACS 126.

For a displayed spectral image, the additional information (e.g., the keV of a mono-energetic image) can be changed during viewing, where a new spectral image is reconstructed and displayed based on the change. For instance, for a contrast enhanced scan that produces volumetric image data to be evaluated for arterial narrowing by calcified arterial plaque, a lower energy level (e.g., 70 keV) with good iodine contrast can first be entered to generate a spectral image to segment the vessel in the image, and then the energy level can be changed to a higher energy level (e.g., 100 keV) to generate a spectral image which better visualizes calcified arterial plaque in the segmented vessel. The level can be changed by entering a specific level via an input field (e.g, FIG. 4, the input box 404), selecting a level from a list of available levels (e.g, FIG. 3, the list 304), sliding a slider 602 along a bar 604 of a selection widget 606 (e.g, FIG. 6), turning a knob 702 of a selection widget 704 (e.g, FIG. 7), a voice command, eye tracking, etc. In one instance, the initial image displayed may be a non-spectral image that is derived from the spectral images and/or a default spectral image.

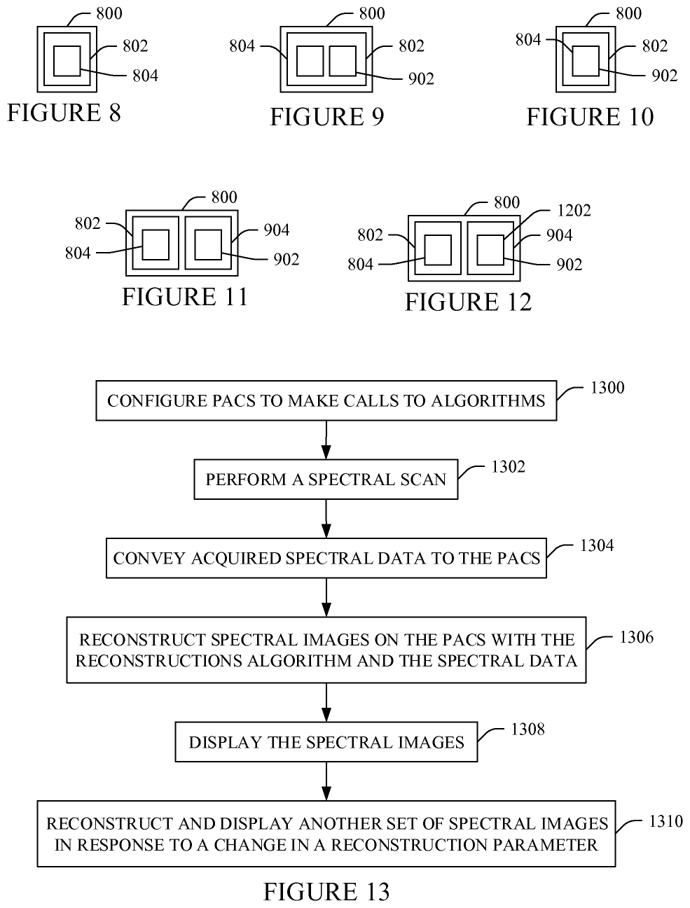

For display, a GUI 800 may include a single viewport 802 with a single image 804 (FIG. 8), the single viewport 802 with multiple images 804, 902, . . . displayed in a split screen format (horizontal (as shown), vertical, diagonal, etc.) with no overlap (FIG. 9), the single viewport 802 with the multiple images 804, 902, . . . partially or fully (as shown) overlapping with a transparency, color and/or other setting to concurrently view information presented in both images (FIG. 10), multiple viewports 802, 904, . . . each displaying a different single image 804 and 902 (FIG. 11), the multiple viewports 802, 904, . . . where at least one viewport (904 in this example) displays multiple images 902 and 1202, e.g., with partial, full (shown) or no overlap with a transparency, color and/or other setting to concurrently view information presented in both images (FIG. 12), etc. Where more than one image is visually displayed, the images can be linked together such as an object identified in one image can be located in another image through the link. For example, the link may include a one-to-one mapping of pixels/voxels amongst the displayed images.

In the above example, the spectral imaging data in the sdata 138 (FIG. 1) and the spectral reconstruction algorithms 144 (FIG. 1) are provided by the same vendor, which is the vendor of the imaging system 102 (FIG. 1). In a variation, the spectral imaging data in the sdata 138 (FIG. 1) is provided by the vendor of the imaging system 102 and the spectral reconstruction algorithms 144 (FIG. 1) are provided by a different vendor, which is also different from the vendor of the PACS 126 (FIG. 1). In another variation, the spectral reconstruction algorithms 144 (FIG. 1) are provided by the vendor of the imaging system 102 (FIG. 1) and the spectral imaging data in the sdata 138 (FIG. 1) is provided by a different vendor, which is also different from the vendor of the PACS 126 (FIG. 1).

FIG. 13 illustrates an example method in accordance with an embodiment(s) described herein.

It is to be appreciated that the ordering of the acts in the method is not limiting. As such, other orderings are contemplated herein. In addition, one or more acts may be omitted and/or one or more additional acts may be included.

At 1300, application software of a PACS is modified to make calls to a library of spectral reconstruction algorithms, which are not part of the application software of the PACS but which are stored therein, and display and manipulate spectral images generated with the spectral reconstruction algorithms.

At 1302, a spectral CT scan is performed with an imaging system.

At 1304, the acquired spectral data is conveyed to and stored on the PACS.

At 1306, the application software of the PACS accesses one or more of the spectral reconstruction algorithms and the spectral imaging data stored on the PACS through an API to reconstruct a set of spectral images.

At 1308, the application software of the PACS displays the set of reconstructed spectral images.

At 1310, in response to receiving an input that affects visualization of the spectral images, the application software of the PACS reconstructs and displays another set of spectral images.

The above may be implemented by way of computer readable instructions, encoded or embedded on computer readable storage medium (which excludes transitory medium), which, when executed by a computer processor(s) (e.g., central processing unit (cpu), microprocessor, etc.), cause the processor(s) to carry out acts described herein. Additionally, or alternatively, at least one of the computer readable instructions is carried by a signal, carrier wave or other transitory medium, which is not computer readable storage medium.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A spectral imaging visualization system, comprising:
a memory device configured to store image visualization application software, spectral imaging data, and spectral image reconstruction algorithms,
wherein the image visualization application software is configured to read electronic files containing images and formatted in a first format, the spectral imaging data is formatted in a second different format, which the image visualization application software cannot read and/or interpret, and the spectral image reconstruction algorithms are configured to read electronic files formatted in the second different format;
a processor configured to access at least one of the spectral image reconstruction algorithms through a proprietary software interface and process the spectral imaging data with the at least one of the spectral image reconstruction algorithms to produce a spectral image, and configured to execute the image visualization application software to construct a graphical user interface with an image viewport displaying the spectral image; and
a display configured to display the graphical user interface with the spectral image displayed in the viewport.

2. The system of claim 1, wherein the spectral imaging data is formatted in a proprietary Digital Imaging and Communications in Medicine format.

3. The system of claim 1, wherein the memory device is further configured to store non-spectral imaging data formatted in the first format.

4. The system of claim 1, wherein the spectral image reconstruction algorithms include at least one of: a mono-energetic algorithm, a calcium suppression algorithm, an effective Z algorithm, an electron density algorithm, an iodine density algorithm, an iodine suppressed algorithm, a contrast-enhanced algorithm, a virtual non-contrast algorithm, a uric acid algorithm, a uric acid suppression algorithm, a basis pair algorithm, and a K-edge algorithm.

5. The system of claim 1, wherein the displayed graphical user interface includes a menu with a plurality of individual selectable graphical control elements, each having a label with a different type of spectral processing, and each configured to call a spectral image reconstruction algorithm of the spectral image reconstruction algorithms corresponding to the label.

6. The system of claim 5, wherein at least one of the plurality of individual selectable graphical control elements is configured to activate a dialog box in response to being selected.

7. The system of claim 6, wherein the dialog box includes a character input field configured to receive a parameter employed by the spectral image reconstruction algorithm.

8. The system of claim 7, wherein the dialog box further includes a second character input field configured to receive a second parameter, and the first and second parameters are employed by the spectral image reconstruction algorithm.

9. The system of claim 6, wherein the dialog box includes a list of selectable parameters, such that one of the parameters selected from the list is employed by the spectral image reconstruction algorithm.

10. The system of claim 7, wherein the displayed graphical user interface includes a widget configured to set a new value of the parameter after the spectral image is displayed in the viewport, and the processor is configured to process the spectral imaging data with the least one of the spectral image reconstruction algorithms and the new value to produce a new spectral image, which is displayed in the image viewport.

11. The system of claim 1, wherein the graphical user interface includes a plurality of image viewports, each configured to display a different spectral image.

12. The system of claim 1, wherein the image viewport is configured to display at least one other spectral image in a split screen format.

13. The system of claim 1, wherein the image viewport is configured to display at least a sub-portion of one spectral image superimposed over another spectral image.

14. A non-transitory computer readable medium encoded with computer executable instructions which, when executed by a processor, cause the processor to perform a method for spectral imaging visualization, comprising:
receiving spectral image reconstruction algorithms and spectral imaging data;
accessing at least one of the spectral image reconstruction algorithms through a proprietary software interface;
processing the spectral imaging data with the at least one of the spectral image reconstruction algorithms to produce a spectral image;
executing image visualization application software to construct a graphical user interface with an image viewport displaying the spectral image, wherein the image visualization application software cannot read and/or interpret the spectral imaging data; and
displaying the graphical user interface with the spectral image displayed in the viewport.

15. The computer readable medium of claim 14, wherein the spectral imaging data is formatted in a proprietary Digital Imaging and Communications in Medicine format.

16. The computer readable medium of claim 14, wherein
the spectral image reconstruction algorithms include at least one of: a mono-energetic algorithm, a calcium suppression algorithm, an effective Z algorithm, an electron density algorithm, an iodine density algorithm, an iodine suppressed algorithm, a contrast-enhanced algorithm, a virtual non-contrast algorithm, a uric acid algorithm, a uric acid suppression algorithm, a basis pair algorithm, and a K-edge algorithm.

17. The computer readable medium of claim 14, wherein
the computer readable medium is part of a hardware memory device of a visualization computing system or an image analyzing/processing computing system.

18. A method for spectral imaging visualization, comprising:
receiving proprietary spectral image reconstruction algorithms and proprietary spectral imaging data at a picture archive and communication system;
accessing, with the picture archive and communication system, at least one of the proprietary spectral image reconstruction algorithms only through a proprietary software interface;
processing, with the picture archive and communication system, the proprietary spectral imaging data with the at least one of the proprietary spectral image reconstruction algorithms to produce a spectral image;
executing, with the picture archive and communication system, image visualization application software of the picture archive and communication system to construct a graphical user interface with an image viewport displaying the spectral image, wherein the image visualization application software cannot read and/or interpret a format of the proprietary spectral imaging data; and
displaying the graphical user interface with the spectral image displayed in the viewport.

19. The method of claim 18, wherein the spectral imaging data is formatted in a proprietary Digital Imaging and Communications in Medicine format.

20. The method of claim 19, wherein the spectral image reconstruction algorithms include at least one of: a mono-energetic algorithm, a calcium suppression algorithm, an effective Z algorithm, an electron density algorithm, an iodine density algorithm, an iodine suppressed algorithm, a contrast-enhanced algorithm, a virtual non-contrast algorithm, a uric acid algorithm, a uric acid suppression algorithm, a basis pair algorithm, and a K-edge algorithm.
